(19) United States
(12) Patent Application Publication
(10) Pub. No.: US 2024/0255374 A1
(43) Pub. Date: Aug. 1, 2024

(54) GAS ANALYZER APPARATUS

(71) Applicant: Crowcon Detection Instruments Limited, Abingdon (GB)

(72) Inventor: Paul Basham, Abingdon (GB)

(21) Appl. No.: 18/419,982

(22) Filed: Jan. 23, 2024

(30) Foreign Application Priority Data
Jan. 26, 2023 (GB) 2301144.8

(51) Int. Cl. G01M 3/16; G01N 33/00

(52) U.S. Cl. CPC G01M 3/16; G01N 33/004; G01N 33/005

(57) ABSTRACT

A gas analyzer apparatus configured to detect the presence of one or more target gases, the gas analyzer apparatus comprising a first gas probe configured to measure one or more flue gases; a second gas probe configured to measure the presence of one or more refrigerant gas; a gas detection unit which selectively communicates with the first and second gas probes to detect the presence of a target gas; wherein the gas detection unit comprises a housing containing a power source and a user interface, wherein the user interface is configured to allow a user to selectively operate, enable and disable the first flue gas detection unit and the second refrigerant gas detection unit; and wherein in use the first and second gas probes are powered by the same power source.

### CROSS-REFERENCE TO RELATED APPLICATIONS

This application claims priority to GB Patent Application Serial No. 2301144.8, filed Jan. 26, 2023, entitled “Gas Analyzer Apparatus,” the entire disclosure of which is hereby incorporated by reference in its entirety.

### TECHNICAL FIELD

The present invention relates to improvements in gas detector technology, in particular detectors used by heating engineers and technicians.

### BACKGROUND

It is known for heating engineers, to work on a variety of different environmental control systems. Each system e.g., heating and air conditioning, has different requirements and equipment that is used in order to service, repair, and maintain such systems.

This may require the engineer to carry multiple separate devices which can increase cost. Accordingly, there is a desire for a lower cost solution.

### SUMMARY OF INVENTION

In order to mitigate at least some of the issues known with the prior art there is provided.

A gas analyzer apparatus configured to detect the presence of one or more target gases, the gas analyzer apparatus comprising a first gas probe configured to measure one or more flue gases; a second gas probe configured to measure the presence of one or more refrigerant gas; a gas detection unit which selectively communicates with the first and second gas probes to detect the presence of a target gas; wherein the gas detection unit comprises a housing containing a power source and a user interface, wherein the user interface is configured to allow a user to selectively operate, enable and disable the first flue gas detection unit and the second refrigerant gas detection unit; and wherein in use the first and second gas probes are powered by the same power source.

The present invention therefore provides an improved device in which costs are reduced as the single device is able to reuse the power supply and user interface for both flue gas analysis and refrigerant gas analysis.

### BRIEF DESCRIPTION OF DRAWINGS

The present disclosure is illustrated by way of example and not limited in the accompanying figures in which like reference numerals indicate similar elements and in which:

FIG. 1 is a side elevation of a gas detector in accordance with an embodiment of the invention; and

FIG. 2 is an example of gas probe in accordance with an embodiment of the invention.

### DETAILED DESCRIPTION

The present invention provides a device for detecting the presence of one more target gases. A gas analyzer apparatus configured to detect the presence of one or more target gases, the gas analyzer apparatus comprising: a first gas probe configured to measure one or more flue gases; a second gas probe configured to measure the presence of one or more refrigerant gas; a gas detection unit which selectively communicates with the first and second gas probes to detect the presence of a target gas; wherein the gas detection unit comprises a housing containing a power source and a user interface, wherein the user interface is configured to allow a user to selectively operate, enable and disable the first flue gas detection unit and the second refrigerant gas detection unit; and wherein in use the first and second gas probes are powered by the same power source.

The target gas is one or more gases which are to be identified and detected. Examples of target flue gases includes oxygen, carbon dioxide, nitrogen monoxide, carbon monoxide. Examples of refrigerant gases includes fluorocarbons such as Chlorofluorocarbons, hydrochlorofluorocarbons, hydrofluoroolefins, etc. The apparatus comprises a gas detection unit which comprises, or can be connected to, a number of gas probes. The probes are inserted into the environment to be tested, such a flue, or refrigerant system, and detect the presence of the target gas within the system or leaks around it. The gas detector utilises the readings from the first and/or second probe to determine the presence, or absence, of the target gas in the environment. Beneficially, the device enables for the selective enablement and disablement of each probe. As the apparatus shares the same housing and interface the device is cheaper to manufacture and more practical for the end user to utilise as the increased functionality of the device reduces the amount of measuring equipment required.

In a development, the housing a ruggedised housing.

Gas detectors are often used in harsh environments. The ruggedised housing provides protection to the apparatus and further allows the apparatus to be used in multiple different environmental conditions.

In a development, the user interface is situated on or in the housing. In a development, the user interface is configured to output a reading indicative of the detection of one or more target gases. In a development, the user interface comprises a display and a user interface input means. In a development, the user interface input means comprises one of a touchscreen interface and/or buttons.

As the apparatus utilises a common interface to control the probe having the interface in the housing with the above-mentioned functionality ensures that the device remains compact and easy to use.

In a development, the first gas probe is a flue gas probe. In a development, the flue gas probe is configured to measure the presence of one or more of: oxygen, carbon monoxide.

For heating engineers, the detection of flue gases is often important in the repair and maintenance of systems they typically encounter.

In a development, the second gas probe is a refrigerant leak probe. In a development, the refrigerant leak probe is a metal Oxide Semiconductor sensor—MOS sensor—or an Infrared sensor—IR sensors.

A heating engineer may also work on air conditioning systems as well as flue systems. The ability to detect such gases is important during their normal course of business. The MOS and IR sensors are particular advantageous given their sensitivity to the concentrations of the target gases.

In a development, the refrigerant leak probe is configured to measure the presence of one or more fluorocarbon gases.

In a development, the gas detection unit comprises a first port configured to interface with the first and/or second gas probes. In a development, the port is a female port configured to interface with a male connector on the first and/or second gas probes.

The functionality of the unit increases with the ability of the end user to connect and disconnect probes as required. This provides for a more versatile device that may be used in multiple different use case scenarios.

In a development, the gas detection unit comprises wireless communication means configured to enable the gas detection unit to communicate with the first and/or second gas probes. In a development, the gas detection unit comprises communication means configured to enable the gas detection unit to communicate with an external computing device and/or the internet.

Often it is beneficial to have the gas detector to connect to external computing devices. For example, where the device is used across a work force being able to monitor and detect exposure to gases is advantageous.

An embodiment of the present invention will now be described with reference to the attached figures. It is to be noted that the following description is merely used for enabling the skilled person to understand the present invention, without any intention to limit the applicability of the present invention to other embodiments which could be readily understood and/or envisaged by the reader. In particular, the present invention is described in relation to gas burners and refrigerant systems, such as domestic systems. However, it will be appreciated that the present invention could be applied in other areas such as in the industrial and commercial context. Furthermore, whilst the device is taught with specific reference to a handheld device the techniques described herein may be utilised for other forms of device, such as mounted devices.

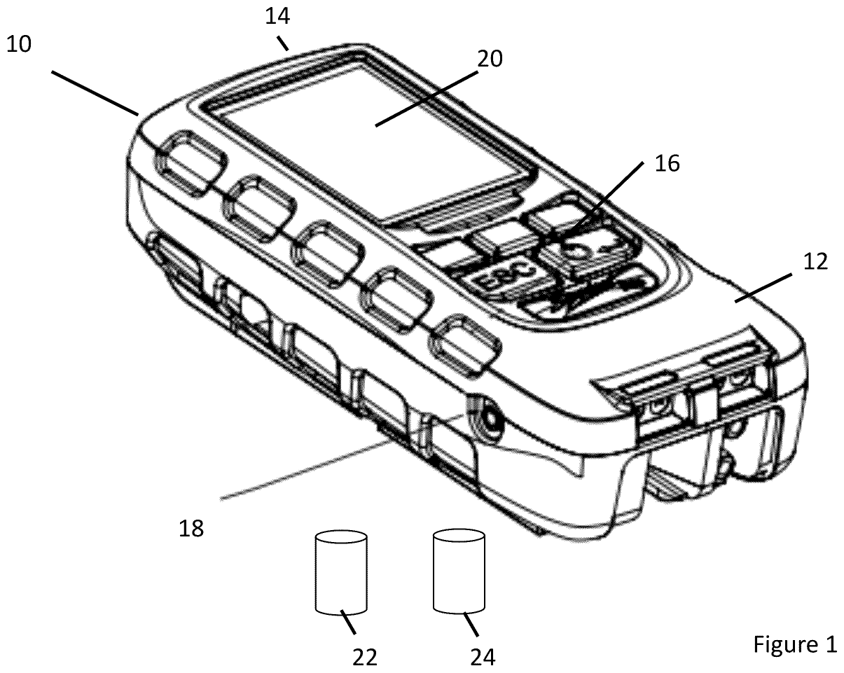

FIG. 1 is an elevation view of a gas analyzer apparatus in accordance with the invention.

There is shown the apparatus 10, comprising a gas detection unit 12, a housing 14, a user interface 16, a port 18, a display 20, first gas probe 22 and a second gas probe 24. The gas detection unit 12 further comprises a power source (not shown in FIG. 1).

The apparatus 10 comprises a gas detection unit 12 which is enclosed in a housing 14. Preferably, the housing 14 is rugged, or ruggedised housing. The gas detection unit 12 is a known gas sensor and configured to the presence and preferably the concentration of one or more target gasses.

Preferably, the gas detection unit 12 further comprises computing elements such as a processor, a memory, and a transmitter. The gas detection unit 12 preferably further comprises a form of memory, preferably non-volatile memory which is capable of storing data when the gas detector is deactivated.

The gas detection unit 12 comprises a display 20 allowing a user to interact with the device in a known manner. For example, the display 20 may show exposure data to the gas detected and concentrations detected by the probes 20 or 22 in such examples the processor produces the exposure data from the probes in a known manner. Alternatively, as in known gas detection units 12 the display 20 may show options to configure and control a gas detection unit 12 in a known manner. The display 20 may be a known touchscreen interface. In such embodiments the touchscreen interface also functions as the user interface 16.

In an embodiment the first gas probe 22 and second gas probe 24 can be selectively attached and detached to the gas detection unit 12 via port 18. In an embodiment the port 18 is a female port, such as a known jack connector port and the first gas probe 22 and second gas probe 24 have a male connector such as a jack. In further embodiments the gas probes connect and communicate to the gas detection unit 12 via known wireless transmission means, such as Bluetooth.

In a further embodiment the first gas probe and second gas probe are integrated into the gas detection unit 12. In a further embodiment the gas probes are permanently connected to the gas detection unit 12 through wired connections.

The gas detection unit 12 further comprises a user interface 16 configured to allow a user to interact with the device. The user interface 16 may comprise buttons, or an alphanumeric keypad, or a touchscreen interface. Any suitable means for enabling a user to input a command to the gas detection unit 12 can be used as the user interface. The user interface 16 is common to the either gas probe and the interface is used to selectively enable and disable each of the probes via the user interface 16 in a known manner. The user interface 16 is common to both probes therefore provides a lower cost and easier to use device.

The gas detection unit 12 further comprises a power source (not shown). The power source is preferably a rechargeable battery which is contained within the housing 14. In an embodiment the power source is a removable and replaceable lithium ion battery. The power source powers all aspects of the gas detection unit 12 including the probe. As the power source is common to the probes the cost of the device is reduced.

The first gas probe 22 is preferably a flue gas probe. The flue gas probe is a known flue gas probe configured to detect one or more flue gases such as oxygen, carbon monoxide, carbon dioxide, nitrogen, nitrogen monoxide and hydrogen. The first gas probe comprises an element which is insertable into a heating system, such as a boiler.

The second gas probe 24 is preferably a refrigerant gas probe. The refrigerant gas probe is a known gas probe configured to detect one or more refrigerant gases such as fluorocarbons such as Chlorofluorocarbons, hydrochlorofluorocarbons, hydrofluoroolefins, etc. Preferably given the levels and sensitivity of the refrigerant gases the probe is a metal Oxide Semiconductor sensor—MOS sensor—or an Infrared sensor—IR sensors.

The apparatus 10 therefore provides an easy to use and cheaper to produce gas detection apparatus. The common interface to enable and disable the probes and the common power source and display ensure that the user is able to service a variety of heating and cooling systems with the same device. This increased functionality results in reduced costs and improved usability.

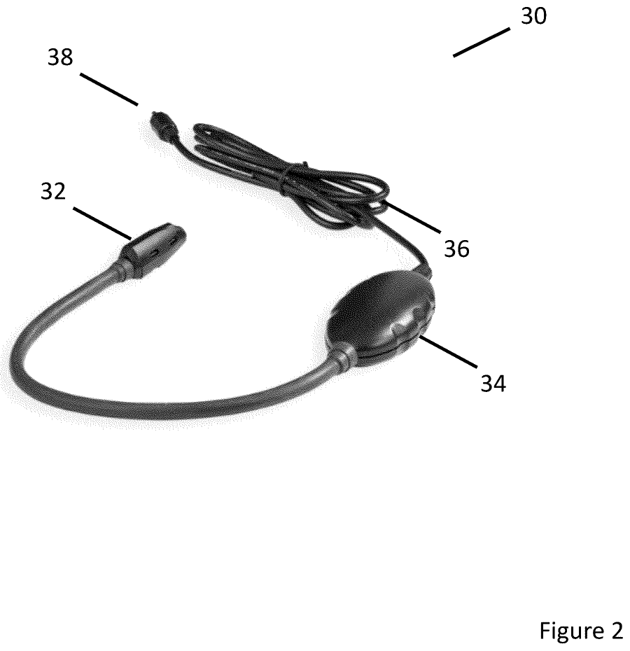

FIG. 2 is an example of gas probe used in an embodiment of the invention.

There is shown the probe 30, having an insertable end 32, a graspable portion 34, cable 36 and a male connector 38.

The gas probe may be any suitable commercially available probe. The probe 30 is insertable by the insertable end 32 into the environment to be tested. The probe 30 is handled and manipulated via the graspable portion 34 and the cable 36 allows for the insertable end 32 to be introduced into an environment to be tested. The probe 30 is connectable to the gas detection unit 12 via the male connector 38 and the port 18.

The apparatus 10 in an embodiment is further configured to communicate with an external computing device, such as a laptop or desktop computer. The apparatus 10 transfers information regarding the user's exposure and usage to the external device to allow for monitoring of the user and the device. In a further embodiment the apparatus 10 is further configured to connect to the internet through known means to affect the communication.

What is claimed is:

1. A gas analyzer apparatus configured to detect the presence of one or more target gases, the gas analyzer apparatus comprising:
a first gas probe configured to measure one or more flue gases;
a second gas probe configured to measure the presence of one or more refrigerant gas; a gas detection unit which selectively communicates with the first and second gas probes to detect the presence of a target gas;
wherein the gas detection unit comprises a housing containing a power source and a user interface, wherein the user interface is configured to allow a user to selectively operate, enable and disable the first and second gas probes; and
wherein in use the first and second gas probes are powered by the same power source.

2. The gas analyzer apparatus of claim 1, wherein the housing a ruggedised housing.

3. The gas analyzer apparatus of claim 1, wherein the user interface is situated on or in the housing.

4. The gas analyzer apparatus of claim 1, wherein the user interface is configured to output a reading indicative of the detection of one or more target gases.

5. The gas analyzer apparatus of claim 1, wherein the user interface comprises a display and a user interface input means.

6. The gas analyzer apparatus of claim 5, wherein the user interface input means comprises one or more of a touchscreen interface, alphanumeric keypad and/or buttons.

7. The gas analyzer apparatus of claim 1, wherein the first gas probe is a flue gas probe.

8. The gas analyzer apparatus of claim 7, wherein the flue gas probe is configured to measure the presence of one or more of: oxygen, carbon monoxide, carbon dioxide, nitrogen monoxide, and hydrogen.

9. The gas analyzer apparatus of claim 1, wherein the second gas probe is a refrigerant leak probe.

10. The gas analyzer apparatus of claim 9, wherein the refrigerant leak probe is a metal Oxide Semiconductor sensor-MOS sensor-or an Infrared sensor-IR sensors.

11. The gas analyzer apparatus of claim 9, wherein the refrigerant leak probe is configured to measure the presence of one or more fluorocarbon gases.

12. The gas analyzer apparatus of claim 1, wherein the gas detection unit comprises a first port configured to interface with the first and/or second gas probes.

13. The gas analyzer apparatus of claim 12, wherein the first port is a female port configured to interface with a male connector on the first and/or second gas probes.

14. The gas analyzer apparatus of claim 1, wherein the gas detection unit comprises wireless communication means configured to enable the gas detection unit to communicate with the first and/or second gas probes.

15. The gas analyzer apparatus of claim 1, wherein the gas detection unit comprises communication means configured to enable the gas detection unit to communicate with an external computing device and/or the internet.
